(19) United States
(12) Patent Application Publication
(10) Pub. No.: US 2017/0202513 A1
(43) Pub. Date: Jul. 20, 2017

(54) IMPLANTABLE PRESSURE SENSORS AND MEDICAL DEVICES

(71) Applicant: THE ALFRED E. MANN FOUNDATION FOR SCIENTIFIC RESEARCH, Santa Clarita, CA (US)

(72) Inventors: Siegmar Schmidt, Simi Valley, CA (US); William A. Dai, Porter Ranch, CA (US)

(73) Assignee: THE ALFRED E. MANN FOUNDATION FOR SCIENTIFIC RESEARCH, Santa Clarita, CA (US)

(21) Appl. No.: 15/374,538

(22) Filed: Dec. 9, 2016

Related U.S. Application Data
(60) Provisional application No. 62/265,102, filed on Dec. 9, 2015.
(60) Provisional application No. 62/315,592, filed on Mar. 30, 2016.

(51) Int. Cl. A61B 5/00; A61N 1/02; A61N 1/05; A61N 1/36; A61B 5/03; A61B 5/08

(52) U.S. Cl. CPC A61B 5/686; A61B 5/036; A61B 5/0803; A61B 5/4836; A61N 1/05; A61N 1/3601; A61N 1/025; A61B 2562/0233; A61B 2562/0261; A61B 2562/0247

(57) ABSTRACT

An implantable pressure sensor device and devices incorporating an implantable pressure sensor are disclosed. The implantable pressure sensor may include a housing with a deflectable wall, and may be incorporated into a housing of an implantable medical device such as an implantable pulse generator. The pressure sensor may monitor respiration by measuring the deflection of the deflectable wall caused by expansion of the thoracic wall or ribcage.

### CROSS-REFERENCE TO RELATED APPLICATION(S)

This application claims priority to and the benefit of U.S. Provisional Application Ser. No. 62/265,102, filed Dec. 9, 2015, the entire contents of which are incorporated herein by reference.

### BACKGROUND

The following Background discussion is only for enhancement of understanding of the background of the invention, and therefore it may contain information that does not form the prior art that is already known to a person of ordinary skill in the art.

Implantable pressure sensors may be used to measure pressures within the body of a patient. They may be used to detect pressures based on respiration, acting as implanted respiration sensors. These implantable pressure sensors may need to be implanted in a respiration-sensitive location, for example, inside the thoracic cavity or inside respiratory muscles such as the intercostal muscles, to usefully or accurately measure respiration.

Implantable medical devices may be used to treat various disorders, including respiration related disorders such as sleep apnea. To account for respiration in these devices, a pressure sensor or other type of breathing sensor may be connected to the implantable medical device. The pressure sensor—implanted at the respiration-sensitive location—may be connected to the implantable medical device using a lead to deliver power to the sensor and to deliver the signal to the implantable medical device. Such leaded sensors may add significant cost and technical complexity to the system. A leaded sensor may also complicate the surgical implant procedure, for example by requiring tunneling of the lead, and increase the risk of medical complication for the patient.

### SUMMARY

According to aspects of embodiments of the present disclosure, a system for detecting a pressure signal is provided.

In one aspect of the present disclosure, a hermetically sealed implantable pressure sensor is provided. The implantable pressure sensor includes a housing having a deflectable wall and enclosing a hermetically sealed cavity, and a deflection sensor configured to measure the deflection of the deflectable wall to generate a pressure signal based on the measured deflection, wherein the housing is configured for implantation in a patient in a thoracic region, outside a rib cage, and the deflectable wall is configured to deflect responsive to pressure exerted on the housing due to respiration of the patient.

In one embodiment, the housing includes a case and a diaphragm, the deflectable wall includes the diaphragm, the case has an opening, and the diaphragm is integrally bonded with the case at the opening to hermetically seal the opening.

In one embodiment, a surface area of the deflectable wall is at least 25% of a surface area of the housing.

In one embodiment, the housing is configured to be implanted between the skin and a surface of a muscle of the patient with the deflectable wall in surface contact with the muscle.

In one embodiment, the housing is configured to be implanted between the skin and a fascia surrounding a muscle of the patient with the deflectable wall in contact with the fascia and in communication with the surface of the muscle.

In one embodiment, the deflection sensor is an optical interferometer, a piezoelectric element, a piezoresistive element, a strain gauge, or a capacitive sensor.

In another aspect of the present disclosure, an implantable pulse generator is provided. The implantable pulse generator includes a biocompatible housing. The biocompatible housing includes a header for interfacing with a proximal end of a lead, and a deflectable diaphragm integral to the biocompatible housing, wherein the housing, including the diaphragm, defines a hermetically sealed cavity. The implantable pulse generator further includes a deflection sensor in the hermetically sealed cavity to detect deflection of the diaphragm, a controller in the hermetically sealed cavity to generate a stimulation signal based on the deflection of the diaphragm, and a stimulation circuit in the hermetically sealed cavity to apply stimulation to the proximal end of the lead interfaced with the header based on the stimulation signal.

In one embodiment, the deflection of the diaphragm corresponds to a pressure on the diaphragm, the controller generates a pressure signal based on the detected deflection of the diaphragm, and the controller generates the stimulation signal based on the pressure signal.

In one embodiment, the diaphragm is configured to face toward a thoracic cavity of a patient when the housing is implanted in the patient.

In one embodiment, the implantable pulse generator further includes a capacitor including a conductive plate parallel to the diaphragm, the conductive plate being a first electrode of the capacitor, the diaphragm includes a conductive material or has a surface lined with a conductive material, the diaphragm is a second electrode of the capacitor, and the deflection sensor is to measure a capacitance of the capacitor.

In one embodiment, the implantable pulse generator further includes a capacitor, wherein the capacitor includes a first conductive plate as a first electrode and a second conductive plate as a second electrode, the second conductive plate being substantially parallel to the first conductive plate, the first conductive plate does not deflect in response to deflection of the diaphragm, the second conductive plate does deflect in response to deflection of the diaphragm, the second conductive plate is electrically isolated from the diaphragm, and the deflection sensor is to measure a capacitance of the capacitor.

In one embodiment, the deflection sensor includes an optical interferometer.

In one embodiment, the deflection sensor includes a piezoelectric element, a piezoresistive element, a strain gauge, or a capacitive sensor.

In one embodiment, the biocompatible housing further includes a case with an opening, and the diaphragm occludes the opening and is bonded to the case at the periphery of the opening.

In one embodiment, the case and the diaphragm are titanium, a titanium alloy, or stainless steel.

In one embodiment, the housing has a broad face facing a first direction and a thin profile facing a second direction substantially perpendicular to the first direction, the wall of the housing is the broad face, and the deflectable diaphragm has an area greater than half of the area of the broad face.

In one embodiment, the diaphragm has a diameter greater than 0.5 inches and a thickness greater than 0.003 inches.

In one embodiment, the implantable pulse generator further includes a lead, the lead having a distal end with one or more electrodes electrically coupled to the proximal end of the lead.

In one embodiment, the implantable pulse generator further includes a sensor circuit coupled to the header to generate biological signals utilizing the lead.

In one embodiment, the housing is configured to be implanted in a thoracic region of a patient, outside the ribcage, the diaphragm is configured to deflect due to respiration, the deflection sensor is configured to generate a respiration signal based on the deflection of the diaphragm, and the stimulation circuit is configured to deliver stimulation based on the respiration signal.

In one embodiment, the implantable pulse generator further includes a lead, the lead having a distal end with one or more electrode electrically coupled to the proximal end of the lead, and wherein the one or more electrode at the distal end of the lead is configured to be implanted proximate to a hypoglossal nerve of the patient.

In one embodiment, the distal end of the lead includes a nerve cuff electrode.

These and other features and aspects of the present invention will be more fully understood when considered with respect to the following detailed description, appended claims, and accompanying drawings.

### BRIEF DESCRIPTION OF THE DRAWINGS

The accompanying drawings, together with the specification, illustrate preferred and example embodiments of the present invention, and, together with the description, serve to explain the principles of the present invention.

FIG. 1 shows an implantable pressure sensor according to embodiments of the present disclosure.

FIG. 2 is a cross section of a pressure sensor according to embodiments of the present disclosure.

FIG. 3 is a diagram of a pressure sensor implanted in a patient according to embodiments of the present disclosure.

FIG. 4A is a diagram showing a cross section of a pressure sensor including a capacitive deflection sensor according to embodiments of the present disclosure.

FIG. 4B is a diagram showing a cross section of a pressure sensor 450 including another capacitive deflection sensor according to embodiments of the present disclosure.

FIG. 4C is a graph depicting a notional relationship between capacitance and pressure during operation of a capacitive deflection sensor according to embodiments of the present disclosure.

FIG. 5A is a diagram showing a cross section of a deflection sensor including an optical deflection sensor according to embodiments of the present disclosure.

FIG. 5B is a diagram showing a deflection sensor including a Fabry-Pérot optical interferometer according to embodiments of the present disclosure.

FIG. 5C is a diagram showing a deflection sensor including a Michelson optical interferometer according to embodiments of the present disclosure.

FIG. 6 is a block diagram of an implantable medical device according to embodiments of the present disclosure.

FIG. 7 is a diagram of an implantable pulse generator according to embodiments of the present disclosure.

### DETAILED DESCRIPTION

In the following detailed description, only certain example embodiments of the present invention are shown and described, by way of illustration. As those skilled in the art would recognize, the invention may be embodied in many different forms and should not be construed as being limited to the embodiments set forth herein. Descriptions of features or aspects within each example embodiment should typically be considered as available for other similar features or aspects in other example embodiments. Like reference numerals designate like elements throughout the specification.

An implantable pressure sensor according to embodiments of the present disclosure may be configured to monitor respiration from a housing implanted in the thoracic region, outside the ribcage. The implantable pressure sensor may include a housing with a deflectable wall positioned against the ribcage, such that the deflectable wall deflects under pressure exerted on the housing by the ribcage during respiration.

In some embodiments, the implantable pressure sensor may be incorporated as part of the housing of an implantable medical device. For example, an implantable pulse generator may incorporate the pressure sensor into the IPG housing, and may use the respiration signal to tailor the stimulation output, without requiring a lead to a remote pressure sensor or implantation in an abnormal spot.

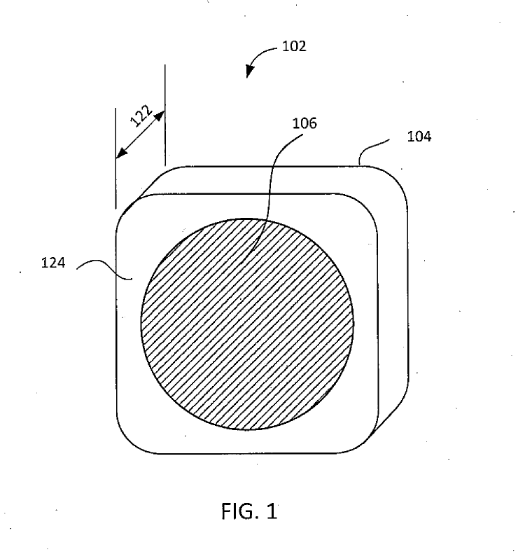

FIG. 1 shows an implantable pressure sensor according to embodiments of the present disclosure. The pressure sensor includes a housing 102. The housing 102 may have a thin profile 122 and a broad face 124. The thin profile 122 may be a side defined by the thickness direction and the length direction of the housing 102, and the broad face 124 may be a side defined by the length direction and the width direction of the housing 102. In some embodiments, the housing 102 may have a substantially cuboid shape as shown in FIG. 1. In other embodiments, the housing may be tapered or rounded off on the side opposite the broad face 124. Although FIG. 1 shows the broad face 124 as substantially rectangular, in other embodiments the broad face 124 may have another suitable shape. The broad face 124 may be substantially flat, or may curve to contour the intended implantation site. For example, the broad face 124 may have a concave curve.

The housing may include a case 104 and a deflectable wall 106. The case 104 may be a rigid case, designed to substantially retain its shape under the pressure levels the pressure sensor is expected to be exposed to. The case 104 may be formed from one or more biocompatible material. In some embodiments, the case 104 may be titanium, stainless steel, or another biocompatible metal alloy. In some embodiments, the case 104 may be a titanium implantable pulse generator case.

The case 104 may form the majority of the housing 102. However, the case 104 may not form the entire housing 102; the case 104 may have a discontinuity. In some embodiments, the discontinuity may be an opening in the case 104, or may be or a region where the characteristics of the housing 102 are different than the characteristics at the case 104. The deflectable wall 106 may form the portion of the housing 102 at the discontinuity. The deflectable wall 106 may be on the broad face 124 of the housing 102.

The deflectable wall 106 may be only a portion of the wall of the housing 102 at the broad face 124, or may be the entire wall at the broad face 124. In some embodiments, the surface of the deflectable wall 106 may be at least 25% of the surface area of the housing 102.

In some embodiments, the deflectable wall 106 is a diaphragm attached at an opening in the case 104. The diaphragm may be integrally bonded to the case 104, occluding the opening. The bond may form a hermetic seal between the diaphragm and the case 104. In some embodiments, the diaphragm is formed from the same material as the case 104, but is thinner than the case. In other embodiments, the diaphragm is formed of a material which is more pliable and/or more elastic than the material of the case 104. The diaphragm and/or the case 104 may be titanium, a titanium alloy, stainless steel, or another biocompatible metal alloy.

For example, in some embodiments the case 104 may be a case for an implantable pulse generator. An opening may be machined into the side of the case 104. A diaphragm may be placed covering the opening, and the diaphragm may be fixed to the case (for example, by laser welding) around the periphery of the opening.

In some other embodiments, the deflectable wall 106 and adjacent portions of the case 104 are formed from a single, continuous element. For example, the deflectable wall 106 and the adjacent portions of the case 104 may be formed of a single piece of metal. The deflectable wall 106 may have characteristics different from the portions of the case 104 which allow it to be deflectable while the case 104 is rigid. For example, in some embodiments, the portion of the piece of metal corresponding to the deflectable wall 106 may be thinner than the portion forming the case 104. In other embodiments, the case 104 may be reinforced and the deflectable wall 106 may not be reinforced or may be less reinforced.

In other embodiments, the case 104 may be configured to bend under force transferred to the case 104 by the deflectable wall 106 when pressure is applied to the broad face 124. Accordingly, the deflectable wall 106 may be deflectable due to the case 104 or certain portions of the case 104 being pliable.

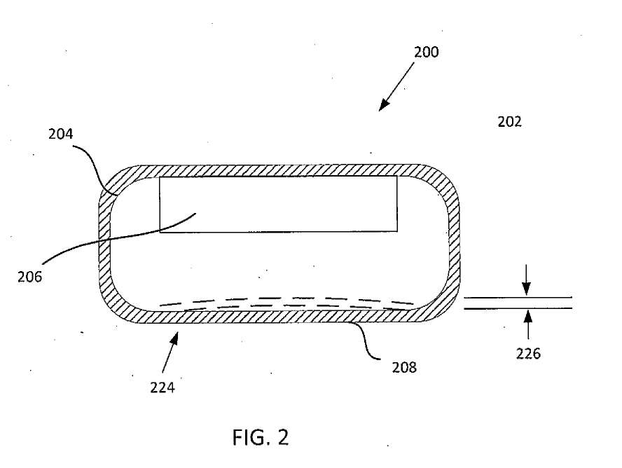

FIG. 2 is a cross section of a pressure sensor 200 according to embodiments of the present disclosure. The housing 202 encloses a hermetically sealed cavity 204. For example, in some embodiments, all portions of the housing 202, such as a case 104 and a deflectable wall 106, are hermetically bonded together, thereby causing the area inside the housing 202 to be hermetically isolated from the area outside the housing 202, such that the cavity inside the housing 202 and defined by the housing 202 is a hermetically sealed cavity.

The pressure sensor 200 may include electronics 206. The electronics 206 may be in the hermetically sealed cavity 204. A deflection sensor may be included in the electronics 206, and may measure the deflection 226 of the deflectable wall 208. The deflection measurement may be correlated with the pressure being exerted on the housing 202. Accordingly, the electronics 206 may generate a pressure signal based on the deflection measurement.

The deflection 226 of the deflectable wall 208 may be based on pressure exerted on the housing 202. A pressure applied in a direction substantially perpendicular or normal to the deflectable wall 208 (e.g. pressure applied to the broad face 224) may cause the deflection 226. Accordingly, the housing 202, the case, and/or the deflectable wall 208 may be designed to have an acceptable deflection in the anticipated pressure ranges. An acceptable deflection 226 may be a deflection that allows the deflection sensor to acquire a deflection measurement with sufficient resolution to determine a respiration signal with sufficient accuracy.

However, as the housing 202 may need to maintain integrity in the anticipated pressure ranges, the housing 202, the case, and the deflectable wall 208 may need to be designed to maintain the integrity of the implantable pressure sensor in the anticipated pressure ranges. Additionally, the, maximum deflection 226 may need to be below a level where the deflectable wall 208 contacts the electronics 206 or other elements contained in the hermetically sealed cavity 204.

In some embodiments, the deflectable wall 208 may be positioned on the broad face 224 of the housing 202. The magnitude of the deflection 226 may be based on the area of the deflectable wall 208. For example, the magnitude of the deflection 226 may be proportional to the fourth power of the size (e.g. lateral dimensions) of the deflectable wall 208. In some embodiments, the deflectable wall 208 may be approximately 0.007 inches thick and 1.2 inches in diameter. In some embodiments, the deflectable wall may be greater than 0.003 inches thick and 0.5 inches in diameter. Positioning the deflectable wall 208 on the broad face 224 of the housing 202 may allow the deflectable wall to have a large enough area that it may be formed of a material which is strong enough, is rigid enough, and/or has a low enough modulus of elasticity to give the housing 202 acceptable integrity, While still allowing the deflection 226 to be of sufficient magnitude to provide an acceptable deflection measurement/pressure signal.

A pressure differential may exist between the internal pressure within the hermetically sealed cavity 204 and the external ambient pressure. In some embodiments, the pressure expected to be applied to the deflectable wall 208 of the housing 202 may be substantially greater than any expected pressure from the pressure differential. Accordingly, the housing 202 and the deflectable wall 208 may be designed such that the deflection 226 occurs based on applied pressure, not pressure differential, and the value of the pressure signal may be substantially independent of the pressure differential.

The magnitude of the deflection 226 resulting from a given pressure applied to the deflectable wall 208 may be influenced by the internal pressure in the hermetically sealed cavity 204. A higher internal pressure within the hermetically sealed cavity may result in a smaller deflection 226 for a given applied pressure. In some embodiments, the internal pressure in the hermetically sealed cavity 204 may be set at a desired level. For example, the ambient pressure at the pressure sensor 200 may be controlled to a desired level at the time of hermetically sealing the housing 202. In some embodiments, the internal pressure is utilized to calibrate the deflection sensor, and the desired level of the internal pressure is the level at which the deflection sensor is calibrated.

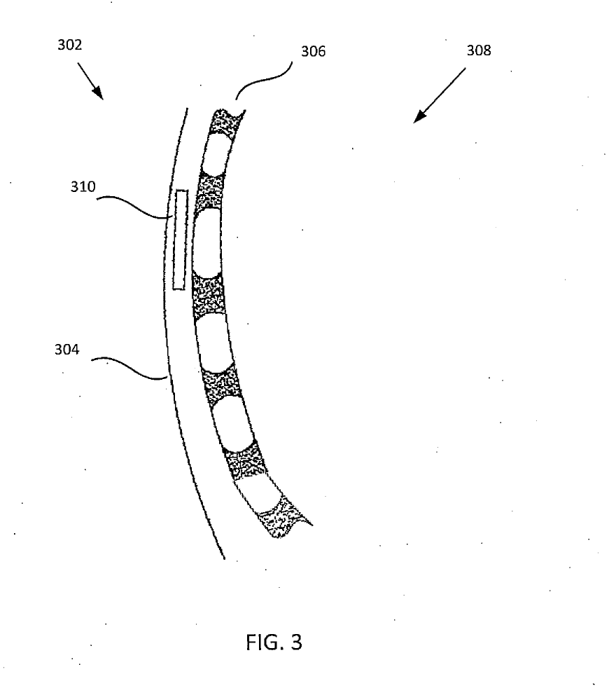

FIG. 3 provides an example embodiment of the pressure sensor. FIG. 3 is a diagram of a pressure sensor 310 implanted in a patient 302 according to embodiments of the present disclosure. The pressure sensor 310 may be the pressure sensor 200 of FIG. 2.

The pressure sensor 310 may be implanted subcutaneously, between the skin 304 and the thoracic wall 306 of a patient. The thoracic wall 306 may be the ribs and muscles (e.g. intercostal muscles) surrounding the thoracic cavity (e.g. the ribcage). The broad face 224, and accordingly the deflectable wall 208, may be facing the thoracic wall 306, on the surface of the thoracic wall 306.

The thoracic wall 306 may apply pressure to the broad face 224 of the pressure sensor 310. When the patient 302 inhales and exhales, the thoracic wall 306 may expand and contract, respectively. The level of pressure applied to the broad face 224 may change based on the degree to which the thoracic wall 306 is expanded or contracted. Accordingly, the deflection 226 of the deflectable wall 208 may vary over time based on the respiration of the patient 302. A pressure signal generated based on a deflection measurement from a deflection sensor may, therefore, be utilized as a signal representing respiration of the patient 302.

In the embodiment of FIG. 3, and referring also to FIG. 2, the housing 202, the case, and/or the deflectable wall 208 of the pressure sensor 310 may be configured to maintain the integrity of the housing 202 based on the pressures expected in the in vivo environment and expected from the thoracic wall 306 pressing on the deflectable wall 308. In some embodiments, they may also be configured to maintain the integrity of the housing under pressure from external sources which may be encountered in day-to-day use, e.g. external application of force on the skin 304 of the patient 302. The housing 202, the case, and/or the deflectable wall 208 may be further configured to deflect under the pressure exerted on the deflectable wall 208 by the thoracic wall 306 during respiration with a sufficient magnitude to acquire a respiration signal. For example, in some embodiments, the deflectable wall 208 may be formed of titanium, may have a thickness of approximately 0.007 inches, and may have a diameter of approximately 1.2 inches. This arrangement may allow the deflectable wall of the housing, when under the pressure of the thoracic wall of the patient pressing on the deflectable wall, to have a deflection of sufficient magnitude to allow the deflection sensor to generate a pressure signal which represents respiration with acceptable accuracy.

Referring again to the pressure sensor 200 of FIG. 2, in some embodiments, the deflection sensor may be mounted on a wall of the housing 202 opposite the deflectable wall 208 and may measure the distance to the deflectable wall 208. In some other embodiments, the housing 202 may include a second deflectable wall opposite the deflectable wall 208 and the deflection sensor may measure the distance between the two deflectable walls. For example, the deflection sensor may be mounted on one deflectable wall and may measure the distance to the other deflectable wall.

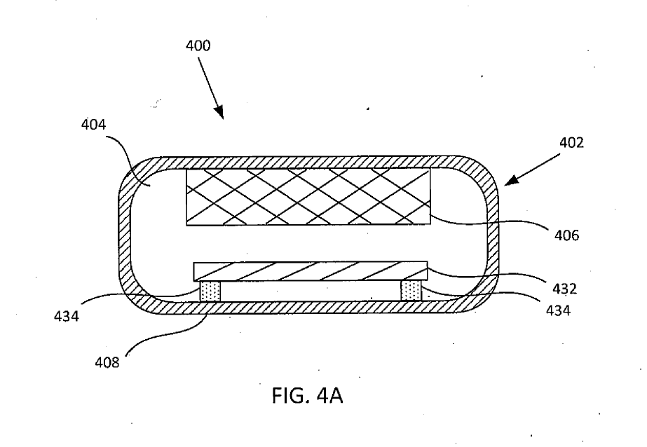

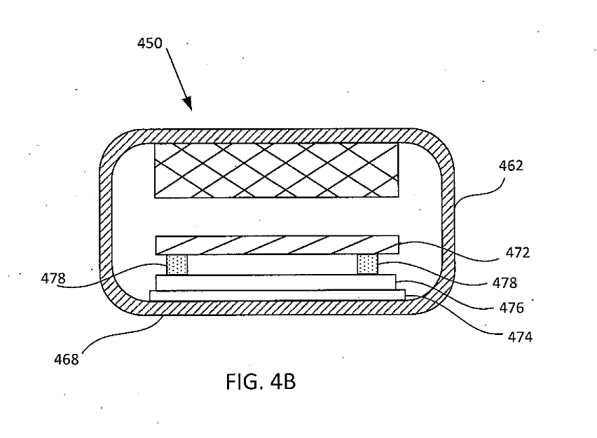

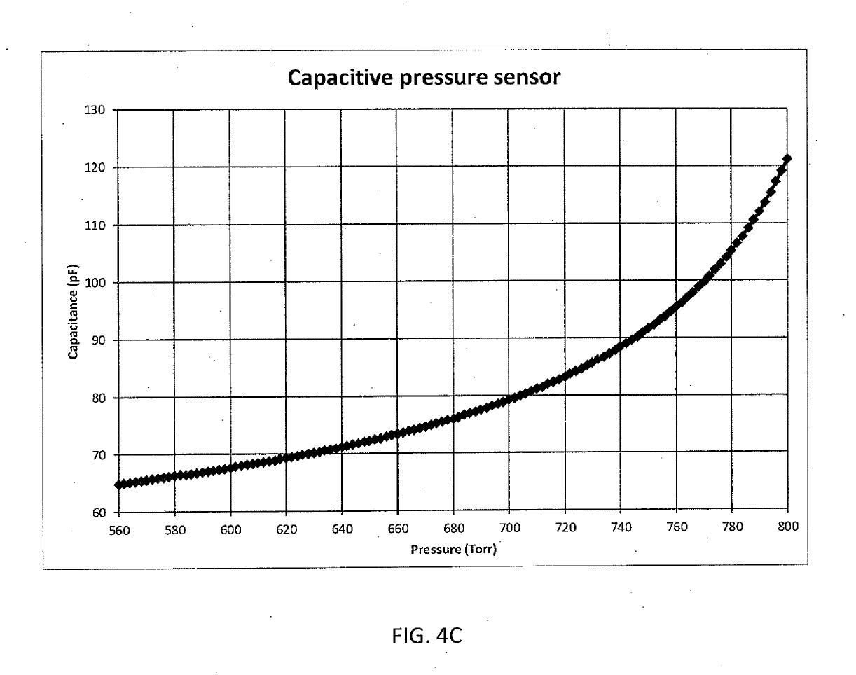

FIG. 4A is a diagram showing a cross section of a pressure sensor 400 including a capacitive deflection sensor according to embodiments of the present disclosure. The deflectable wall 408 may be formed partially or entirely from a conductive material, or may be coated in a conductive material. A conductive plate 432 may be mounted in the hermetically sealed cavity 404 inside the housing 402, spaced apart from the deflectable wall 408 by one or more spacers 434. The one or more spacers 434 may be formed of an electrically insulating material, and may be small and/or,thin. Accordingly, the conductive plate 432 may not be in electrical contact with the deflectable wall 408.

The conductive plate 432 and the conductive deflectable wall 408 may be coupled to the electronics 406. The electronics 406 may measure the capacitance between the conductive plate 432 and the deflectable wall 408. The measured capacitance may be utilized as the deflection measurement.

FIG. 4B is a diagram showing a cross section of a pressure sensor 450 including another capacitive deflection sensor according to embodiments of the present disclosure. In the capacitive deflection sensor of 4B, an insulator 474 is mounted on an inside surface of a deflectable wall 468. The insulator 474 may be a sheet or coating covering a portion of the inside surface of the deflectable wall 468 or the entire inside surface of the deflectable wall 468. The insulator 474 may deflect with the deflectable wall 468.

A lower conductive plate 476 may be coupled to the other side of the insulator 474, and may also deflect with the insulator 474 and the deflectable wall 468. An upper conductive plate 472 may be fixed relative to the housing (or may otherwise not deflect with the deflectable wall 468) in proximity to but spaced apart from the lower conductive plate 476, and not in electrical contact with the lower conductive plate 476. In some embodiments, the upper conductive plate 472 is separated from the lower conductive plate 476 by one or more spacers 478.

The upper conductive plate 472 and the lower conductive plate 476 may form a capacitor in a capacitive deflection sensor. The insulator 474 may electrically isolate the capacitor of the capacitive deflection sensor from the housing 462, which may be a desired feature of the device. For example, where the pressure sensor 450 is implanted subcutaneously, contact with the surrounding tissue may not have a grounding effect on either plate of the capacitive deflection sensor.

FIG. 4C is a graph depicting a notional relationship between capacitance and pressure during operation of a capacitive deflection sensor according to embodiments of the present disclosure. The capacitive deflection sensor may be a deflection sensor as described above in reference to FIGS. 4A and 4B. As shown in FIG. 4C, the capacitance of the capacitive deflection sensor may increase with an increased deflection caused by increased pressure.

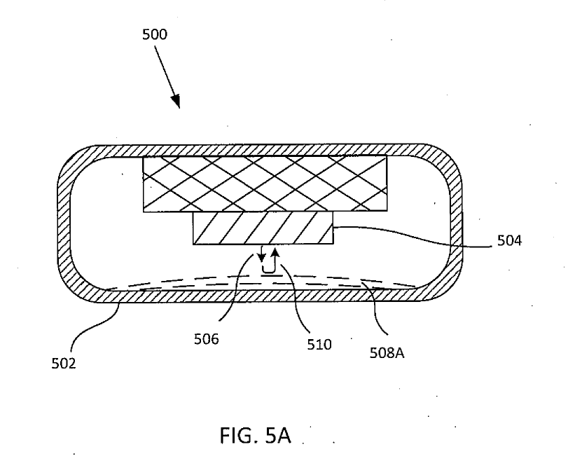

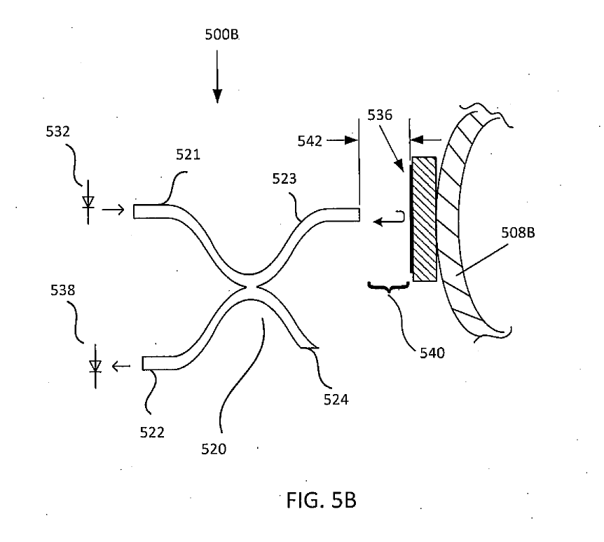

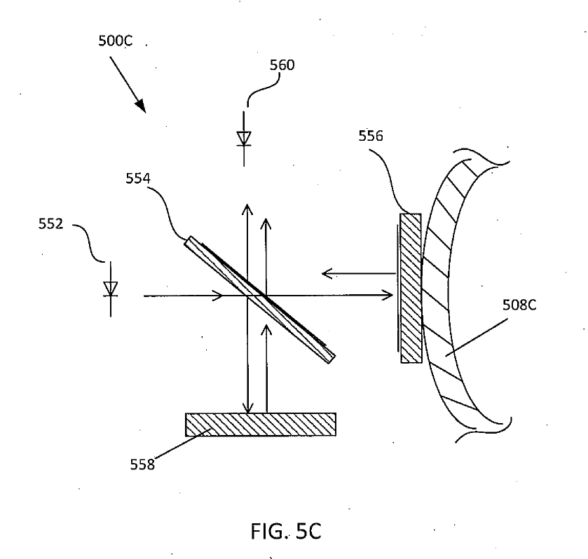

In some other embodiments, the deflection sensor may optically measure the distance between the deflection sensor and the deflectable wall. For example, FIGS. 5A-C depict embodiments of deflection sensors utilizing optical sensors to measure the distance to the deflectable wall.

FIG. 5A is a diagram showing a cross section of a pressure sensor including an optical deflection sensor 504 according to embodiments of the present disclosure. The optical deflection sensor 504 may be mounted on, or otherwise fixed relative to, the housing 502 opposite the deflectable wall 508A and may measure the distance between the optical sensor 504 and the deflectable wall 508A.

In some embodiments, the inside of the deflectable wall 508A may be optically polished. In other embodiments, a mirror may be attached to the inside of the deflectable wall 508A. The optical sensor 504 may send out emitted light 506 toward the deflectable wall 508A. In some embodiments, the optical sensor 504 may utilize an LED to generate the emitted light 506. In other embodiments, the optical sensor 504 may utilize a laser diode to generate the emitted light 506.

The emitted light 506 may be reflected back toward the optical sensor 504 (e.g., the optically polished surface or the mirror may reflect the emitted light 506). The reflected light 510 is utilized to determine the amount of deflection of the deflectable wall 508A.

FIG. 5B is a diagram showing an exemplary deflection sensor 500B including a Fabry-Pérot optical interferometer according to embodiments of the present disclosure. Fabry-Pérot optical interferometers are well known as devices for measuring changes in distance. Deflection sensor 500B is a fiber optic based Fabry-Pérot optical interferometer.

The deflection sensor 500B includes two fiber optic cables interconnected by a fiber optic coupler 520 and has four ports 521, 522, 523 and 524. Optical emitter 532 is coupled to port 521. Optical receiver 538 is coupled to port 522. Port 524 is a terminated end of the sensor 500B. Port 523 has a partially reflective mirror at its end and is facing mirrored surface 536. As pressure is exerted on diaphragm 508B and diaphragm 508B is deflected, the distance 542 in resonator 540 between the end of port 523 and mirror 536 will change and such a change will generate a change in the optical signal received at port 522 and detected by receiver 538. Associated electronics (not shown) coupled to receiver 538 can interpret the change in the optical signal received as a deflection sensed by deflection sensor 500B. The receiver 538 may be a semiconductor PIN-Diode which is sensitive to the wavelength of the emitter 532.

FIG. 5C is a diagram showing a deflection sensor 500C including a Michelson optical interferometer according to embodiments of the present disclosure.

A first mirror 556 may be attached to the inside surface of the deflectable wall 508C. A second mirror 558 may have a fixed position relative to the housing. An emitter 552 may send out an optical collimated beam. The emitter 552 may be a laser diode or an LED. A beam splitter 554, fixed relative to the housing and the second mirror 558, may split the optical collimated beam into two beams, one going toward the first mirror 556 and the other going toward the second mirror 558. The first mirror 556 and the second mirror 558 may reflect the beams back to the beam splitter 554, and the beam splitter 554 may combine the beams. The two combined beams may interfere with each other, resulting in an interference signal. The interference signal may be detected by an optical receiver 560.

The intensity of the interference signal may be based on the distances between the beam splitter 554 and the mirrors 556 and 558. Because the beam splitter 554 and the second mirror 558 are fixed, the intensity of the interference signal may be correlated with the distance between the beam splitter 554 and the first mirror 556. Accordingly, the signal received by the optical receiver 560 may be utilized as a deflection measurement or may be utilized to generate a deflection measurement.

In some other embodiments, the deflection sensor may measure mechanical changes resulting from the deflection of the deflectable wall. The deflection sensor may include a strain gauge, a piezoelectric element, or a piezoresistive element coupled to the deflectable wall such that it moves responsive to deflection of the deflectable wall, thereby generating a deflection measurement which can be utilized to generate a pressure signal.

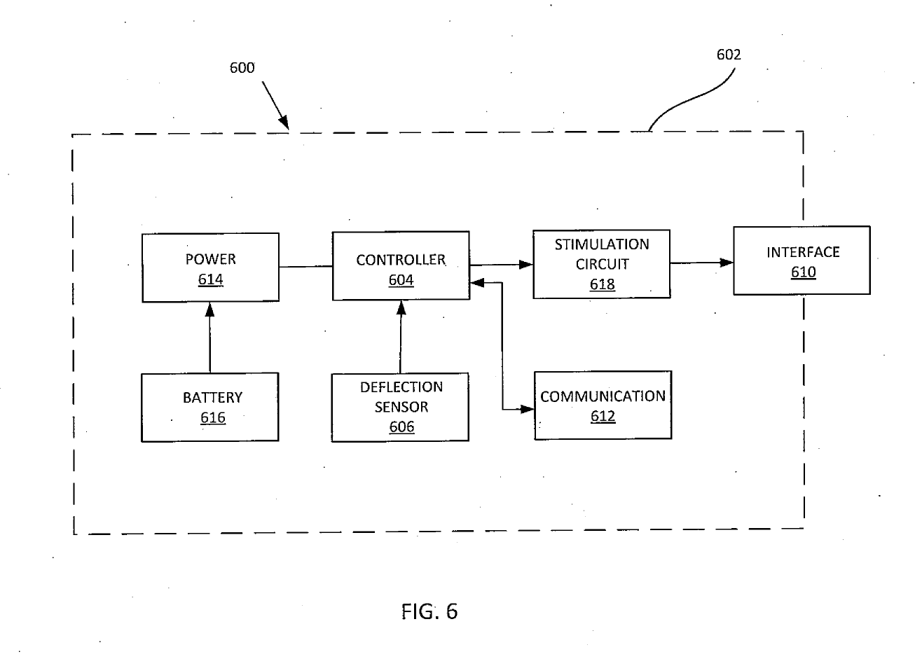

A pressure sensor according to embodiments of the present disclosure may be integrated in a medical device, such as an implantable medical device. FIG. 6 is a block diagram of an implantable medical device 600 according to embodiments of the present disclosure.

The implantable medical device 600 may include a housing 602 enclosing a hermetically sealed cavity for components of the device. For example, the housing 602 may enclose a controller 604. The controller 604 may apply a treatment (such as stimulation) to a patient or determine a treatment to be applied to a patient (e.g. by a stimulation circuit). The controller 604 may be coupled to an interface 610 to deliver the treatment to a patient. The interface 610 may be for applying stimulation, the timing and/or parameters of which are determined inside the housing 602 by the controller 604, to the outside of the housing 602. In some embodiments, the interface 610 may be an electrical feedthrough for electrically coupling with a proximal end of a lead from inside the hermetically sealed housing 602. A header may be coupled to the housing to facilitate electrical coupling between the feedthrough and one or more electrodes of the proximal end of an external lead inserted in the header. A stimulation circuit 618 may be coupled to the controller 604 and the feedthrough. The stimulation circuit 618 may apply stimulation to a patient through electrodes located at the distal end of the lead with parameters determined by the controller 604. In other embodiments, a sensor circuit may be coupled to the external lead through the feedthrough, and the external lead may additionally or alternatively be used to generate biological signals utilizing the electrode or electrodes at a site remote from the implanted medical device 600. In some other embodiments, the interface 610 may be a pump outlet for delivering drugs to a patient.

The controller 604 may additionally or alternatively be coupled to a communication circuit 612 contained inside the housing. The controller 604 may use the communication circuit 612 to transmit information about the patient to an external device, for example as an implantable sensor. The controller 604 may additionally or alternatively use the communication circuit 612 to receive configuration data from an external programmer or to transmit information about treatment applied to a patient, for example as an implantable pulse generator.

The housing 602 may also enclose a power circuit 614. The power circuit 614 may supply power to the controller 604, and/or other components of the implantable medical device 600. In some embodiments, the power circuit 614 may utilize power from a battery 616 and the battery 616 may be a primary battery. In some other embodiments, the power circuit 614 may receive⁻wireless power from an external source, and may use the wireless power to charge the battery 616 or to power the implantable medical device 600 directly.

The housing 602 may include a deflectable wall and a deflection sensor 606. The deflection sensor 606 may be coupled to the controller 604. The deflection sensor 606 may generate a deflection measurement based on deflection of the deflectable wall as described above, and the controller 604 may generate a pressure signal based on the deflection measurement. The controller 604 may utilize the pressure signal in operation of the implantable medical device 600. For example, in some embodiments, the controller 604 may use the pressure signal in determining a treatment to be applied to a patient, such as whether or not to apply stimulation to the patient, when to apply stimulation, and/or what the parameters (e.g. amplitude, frequency, duration) of any applied stimulation should be. In some other embodiments, the controller 604 may communicate the pressure signal to an external device.

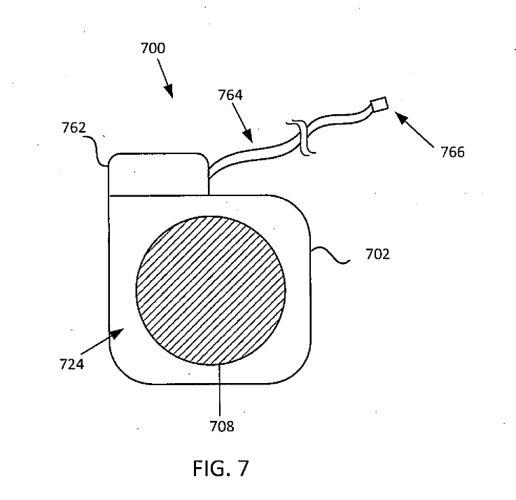

FIG. 7 is a diagram of an implantable pulse generator 700 according to embodiments of the present disclosure. The implantable pulse generator 700 may include a housing 702 which encloses the pulse generator circuitry in a hermetically sealed cavity. The housing 702 may also include a deflectable wall 708 on a broad face 724 of the housing 702, and may enclose a deflection sensor to generate a deflection measurement.

The implantable pulse generator 700 may include a header 762. The header 762 may allow the pulse generator circuitry to electrically couple with an external lead 764 from inside the hermetically sealed chamber of the housing 702. Accordingly, the implantable pulse generator 700 may apply stimulation to a patient via electrodes at a distal end 766 of the external lead 764.

The implantable pulse generator 700 may apply stimulation based on the deflection measurement received from the deflection sensor. For example, in some embodiments, the characteristics of the stimulation (e.g., amplitude, frequency) may be determined based on the deflection measurement. In other embodiments, the implantable pulse generator 700 may apply stimulation during particular intervals, and it may use the deflection measurement to determine when to stimulate.

In an example embodiment, the implantable pulse generator 700 may be configured to be placed in a thoracic region of a patient, outside the ribcage, with the deflectable wall facing the ribcage. This may be as described above in reference to FIG. 3. Accordingly, the deflection measurement may be influenced by respiration. The implantable pulse generator 700 may use the deflection measurement to generate a respiration signal.

The distal end 766 of the external lead 764 may be configured to be implanted at an upper airway muscle or a nerve innervating one or more upper airway muscles. In some embodiments, the distal end 766 may be implanted at the hypoglossal nerve: In some embodiments, the distal end 766 of the external lead 764 may include a nerve cuff electrode. Stimulation applied to the electrodes at the distal end 766 may cause one or more upper airway muscles to contract, or may increase the tonus of one or more upper airway muscles, causing or improving patency of the upper airway of a patient.

The implantable pulse generator 700 may monitor the respiration signal to identify inspiratory periods and expiratory periods of a patient's respiration. The implantable pulse generator 700 may stimulate the muscle or nerve, improving patency of the upper airway, during inspiratory periods but not during expiratory periods. Accordingly, the implantable pulse generator 700 may be used to treat disorders involving obstruction of the upper airway (e.g., Sleep Apnea such as Obstructive Sleep Apnea or Sleep Hypopnea) without unnecessary stimulation, therefore causing less fatigue of the stimulated muscle, and with prolonged battery life and/or reduced power consumption. In some embodiments, the implantable pulse generator 700 may monitor the respiration signal to determine when the patient is asleep, and may apply stimulation when the patient is asleep but not when the patient is awake.

In some embodiments, the implantable pulse generator 700 may include one or more additional sensors within the hermetically sealed chamber of the housing 702, and may use signals from any additional sensor in determining whether to apply stimulation, when to apply stimulation, and/or what the parameters of any applied stimulation may be. Examples of additional sensors include an accelerometer and/or a gyroscope. In some embodiments, the implantable pulse generator 700 uses an accelerometer and/or a gyroscope to determine a posture and/or position of the patient. In the example presented above, where the implantable pulse generator 700 is being used to treat sleep apnea, the implantable pulse generator 700 may use a patient's posture or body position to determine when the patient is laying down, such that stimulation is not applied when the patient is upright, but may be applied when the patient is lying down.

In some embodiments, referring to the implantable pulse generator 700 of FIG.. 7, the pressure signal may monitor the respiration cycle by monitoring changes in the pressure applied to the deflectable wall over time. By monitoring this relative pressure, it may identify inspiratory periods and expiratory periods of the patient's respiration.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present invention.

It will be understood that when an element or layer is referred to as being “on,” “connected to,” or “coupled to” another element or layer, it can be, directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

The terminology used herein is for the purpose of describing particular embodiments and is not intended to be limiting of the present invention. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

As used herein, the term “substantially,” “about,” and similar terms are used as terms of approximation and not as terms of degree, and are intended to account for the inherent variations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively. Also, the term “exemplary” is intended to refer to an example or illustration.

The electronic or electric devices and/or any other relevant devices or components according to embodiments of the present invention described herein may be implemented utilizing any suitable hardware, firmware (e.g. an application-specific integrated circuit), software, or a combination of software, firmware, and hardware. For example, the various components of these devices may be formed on one integrated circuit (IC) chip or on separate IC chips. Further, the various components of these devices may be implemented on a flexible printed circuit film, a tape carrier package (TCP), a printed circuit board (PCB), or formed on one substrate. Further, the various components of these devices may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the spirit and scope of the exemplary embodiments of the present invention.

While this invention has been described in detail with particular references to illustrative embodiments thereof, the embodiments described herein are not intended to be exhaustive or to limit the scope of the invention to the exact forms disclosed. Persons skilled in the art and technology to which this invention pertains will appreciate that alterations and changes in the described structures and methods of assembly and operation can be practiced without meaningfully departing from the principles, spirit, and scope of this invention, as set forth in the following claims and equivalents thereof.

What is claimed is:

1. A hermetically sealed implantable pressure sensor comprising:
a housing having a deflectable wall and enclosing a hermetically sealed cavity; and
a deflection sensor configured to measure the deflection of the deflectable wall to generate a pressure signal based on the measured deflection,
wherein the housing is configured for implantation in a patient in a thoracic region, outside a rib cage, and the deflectable wall is configured to deflect responsive to pressure exerted on the housing due to respiration of the patient.

2. The pressure sensor of claim 1, wherein:
the housing comprises a case and a diaphragm, the deflectable wall comprising the diaphragm,
the case has an opening, and
the diaphragm is integrally bonded with the case at the opening to hermetically seal the opening.

3. The pressure sensor of claim 1, wherein the housing is configured to be implanted between the skin and a surface of a muscle of the patient with the deflectable wall in surface contact with the muscle.

4. The pressure sensor of claim 1, wherein the housing is configured to be implanted between the skin and a fascia surrounding a muscle of the patient with the deflectable wall in contact with the fascia and in communication with a surface of the muscle.

5. The pressure sensor of claim 1, wherein the deflection sensor comprises an optical interferometer, a piezoelectric element, a piezoresistive element, a strain gauge, or a capacitive sensor.

6. An implantable pulse generator comprising:
a biocompatible housing comprising:
a header for interfacing with a proximal end of a lead; and
a deflectable diaphragm integral to, the biocompatible housing, wherein the housing, including the diaphragm, defines a hermetically sealed cavity;
a deflection sensor in the hermetically sealed cavity to detect deflection of the diaphragm;
a controller in the hermetically sealed cavity to generate a stimulation signal based on the deflection of the diaphragm; and
a stimulation circuit in the hermetically sealed cavity to apply stimulation to the proximal end of the lead interfaced with the header based on the stimulation signal.

7. The implantable pulse generator of claim 6, wherein the deflection of the diaphragm corresponds to a pressure on the diaphragm, the controller generates a pressure signal based on the detected deflection of the diaphragm, and the controller generates the stimulation signal based on the pressure signal.

8. The implantable pulse generator of claim 6, wherein the diaphragm is configured to face toward a thoracic cavity of a patient when the housing is implanted in the patient.

9. The implantable pulse generator of claim 6, further comprising a capacitor, wherein:
the capacitor comprises a conductive plate parallel to the diaphragm, the conductive plate being a first electrode of the capacitor,
the diaphragm comprises a conductive material or has a surface lined with a conductive material,
the diaphragm is a second electrode of the capacitor, and
the deflection sensor is to measure a capacitance of the capacitor.

10. The implantable pulse generator of claim 6, further comprising a capacitor, wherein:
the capacitor comprises a first conductive plate as a first electrode and a second conductive plate as a second electrode, the second conductive plate being substantially parallel to the first conductive plate,
the first conductive plate does not deflect in response to deflection of the diaphragm,
the second conductive plate does deflect in response to deflection of the diaphragm,
the second conductive plate is electrically isolated from the diaphragm, and
the deflection sensor is to measure a capacitance of the capacitor.

11. The implantable pulse generator of claim 6, wherein the deflection sensor comprises an optical interferometer.

12. The implantable pulse generator of claim 6, wherein the deflection sensor comprises a piezoelectric element, a piezoresistive element, a strain gauge, or a capacitive sensor.

13. The implantable pulse generator of claim 6, wherein:
the biocompatible housing further comprises a case with an opening, and
the diaphragm occludes the opening and is bonded to the case at the periphery of the opening.

14. The implantable pulse generator of claim 13, wherein the case and the diaphragm are titanium, a titanium alloy, or stainless steel.

15. The implantable pulse generator of claim 6, wherein the housing has a broad face facing a first direction and a thin profile facing a second direction substantially perpendicular to the first direction, and the deflectable diaphragm is integral to the broad face.

16. The implantable pulse generator of claim 6, wherein the diaphragm has a diameter greater than 0.5 inches and a thickness greater than 0.003 inches.

17. The implantable pulse generator of claim 6, further comprising a lead, the lead having a distal end with one or more electrodes electrically coupled to the proximal end of the lead.

18. The implantable pulse generator of claim 17, further comprising a sensor circuit coupled to the header to generate biological signals utilizing the lead.

19. The implantable pulse generator of claim 6, wherein the housing is configured to be implanted in a thoracic region of a patient, outside a ribcage of the patient, the diaphragm is configured to deflect due to respiration, the deflection sensor is configured to generate a respiration signal based on the deflection of the diaphragm, and the stimulation circuit is configured to deliver stimulation based on the respiration signal.

20. The implantable pulse generator of claim 19, further comprising a lead, the lead having a distal end with one or more electrode electrically coupled to the proximal end of the lead, and wherein the one or more electrode at the distal end of the lead is configured to be implanted proximate to a hypoglossal nerve of the patient.

21. The implantable pulse generator of claim 20, wherein the distal end of the lead comprises a nerve cuff electrode.
